(19) United States
(12) Patent Application Publication
(10) Pub. No.: US 2007/0072280 A1
(43) Pub. Date: Mar. 29, 2007

(54) Materials and methods for the efficient production of xylitol

(75) Inventors: Patrick Carmen Cirino, State College, PA (US); Lonnie O'Neal Ingram, Gainesville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: University of Florida Research Foundation, Inc., Gainesville, FL (US)

(21) Appl. No.: 11/523,403

(22) Filed: Sep. 18, 2006

Related U.S. Application Data
(60) Provisional application No. 60/718,411, filed on Sep. 19, 2005.

(51) Int. Cl. C12P 7/18; C12N 1/21; C12N 15/74

(52) U.S. Cl. 435/158; 435/252.33; 435/488

(57) ABSTRACT

Novel microorganisms are provided that efficiently convert xylose (or xylulose) alone or in combination with a carbon substrate to produce xylitol. In certain embodiments, E. coli are engineered to include a mutant crp gene as well as deletion of the xylB gene. The microorganisms of the invention are particularly advantageous because they serve as biocatalysts for the efficient and scalable conversion of biomass-derived sugars into xylitol.

### CROSS-REFERENCE TO RELATED APPLICATION

This application claims the benefit of U.S. Provisional Application Ser. No. 60/718,411, filed Sep. 19, 2005.

### GOVERNMENT SUPPORT

The subject matter of this application has been supported in part by U.S. Government Support under US DOE-DE FG02-96ER200222. Accordingly, the U.S. Government has certain rights in this invention.

### BACKGROUND OF THE INVENTION

Xylitol is a pentahydroxy sugar alcohol found in fruits and vegetables and having sweetness similar to that of sucrose. Parajo, J C et al., “Biotechnological production of xylitol. Part 1: Interest of xylitol and fundamentals of its biosynthesis,” Bioresource Tech, 65:191-201 (1998); and Pepper, T and P M Olinger, “Xylitol in Sugar-Free Confections,” Food Tech, 42:98-106. Xylitol has many favorable properties as a natural, nutritive sweetener and food additive. Most notably, xylitol is noncariogenic and even inhibits the development of dental caries and therefore is used in toothpastes and sugarless confectionaries. In humans, metabolism of this polyol is not insulin-mediated, so xylitol serves as a sugar substitute for diabetics.

Additional auspicious qualities include its large negative heat of dissolution (greater than other sugar substitutes), resulting in a clean, refreshing sensation in the mouth, and its inability to contribute to Maillard-based food browning and caramelization, in contrast to carbonyl-containing sugar substitutes.

Finally, xylitol can serve as a valuable synthetic building block and was recently identified as one of the top twelve value-added materials to be produced from biomass, thereby serving as a key economic driver for biorefineries. For these reasons, it is expected that the demand for xylitol will increase in the future.

Commercial processes for xylitol production primarily use catalytic hydrogenation (reduction) of D-xylose derived from hemicellulose-xylan hydrolysates of biomass materials such as birchwood and corn, where only about 50-60% of the initial xylose is converted to xylitol. Moreover, currently available processes for xylitol production require the use of high pressure (50 atm) and temperatures (80°-140° C.) resulting in low conversion of biomass materials to xylitol. In addition, downstream separation and purification of the resultant xylitol are expensive procedures to implement.

Alternate biological approaches to xylitol production are also being developed. Most reports involve the whole-cell production of xylitol from xylose using various natural or genetically modified yeast strains (see, for example, Nigam, P. and D. Singh, “Processes for Fermentative Production of Xylitol—a Sugar Substitute,” Process Biochem, 30:117-124 (1995); and Parajo, J C et al., “Biotechnological production of xylitol. Part 2: Operation in culture media made with commercial sugars,” Bioresource Tech, 65:203-212 (1998)), although in vitro biocatalytic processes requiring cofactor regeneration systems are also being developed (see for example, Jang, S H et al., “Complete in vitro conversion of D-xylose to xylitol by coupling xylose reductase and formate dehydrogenase,” J Microbiology and Biotech, 13:501-508 (2003) and Suzuki, S. et al., “Novel enzymatic method for the production of xylitol from D-arabitol by Gluconobacter oxydans,” Bioscience Biotech and Biochem, 66:2614-2620 (2002)). Unfortunately, current reported biotransformation methods require multiple-step synthesis and/or have low yields.

The first two steps of D-xylose assimilation in yeasts involve xylose reduction to xylitol via xylose reductase (XR, also called aldose or aldehyde reductase; alditol:NAD(P)+ 1-oxidoreductase, EC 1.1.1.21) followed by xylitol oxidation to xylulose via xylitol dehydrogenase (XDH, also called D-xylulose reductase, xylitol:NAD+ 2-oxidoreductase, EC 1.1.1.9). XR typically prefers NADPH while XDH utilizes NAD+, and it is this propensity for cofactor imbalance in many strains that ultimately leads to xylitol secretion rather than continued metabolism. Further, obtaining the substrate, D-xylose, in a form suitable for yeast fermentation is a considerable problem because inexpensive xylose sources such as sulphite liquor from pulp and paper processes contain impurities that inhibit yeast growth.

Although some bacteria are naturally capable of synthesizing xylitol (see, for example, Yoshitake, J. et al., “Xylitol Production by an Enterobacter Species,” Agricult and Biological Chem, 37:2261-2267 (1973)), few research efforts have focused on using bacteria for xylitol production. Escherichia coli (E. coli) has proven to be a suitable host for the overproduction of a variety of native metabolites as well as non-native compounds. E. coli is an attractive target for metabolic engineering due to the wealth of physiological, metabolic, genetic and regulatory information available, and well-established genetic methods with predictable genetic engineering results. E. coli is also an ideal organism for industrial production of chemicals due to its ability to assimilate both hexose and pentose sugars, rapid growth rates, ease of manipulation, and inexpensive growth medium requirements, as evidenced by its industrial implementation for production of 1,3-propanediol (Nakamura, C E and G M Whited, “Metabolic engineering for the microbial production of 1,3-propanediol,” Curr Opinion in Biotech, 14:454-459 (2003)) and 3-hydroxypropionic acid (Cameron, D C, “3-Hydroxypropionic Acid: a New Platform Chemical from the Biorefinery,” 10^{th} Ann Mtg, Inst. Of Biol Engineering, Athens, Ga., 2005)).

In E. coli, xylose uptake occurs primarily through a high-affinity, ATP-binding cassette (ABC) transporter (XylFGH), even in the presence of high xylose concentrations. A second, low-affinity proton symporter (XylE) is also present but does not appear to play a significant role in xylose transport. The efficiency of xylose utilization in E. coli is therefore suboptimal due to energetic requirements for nutrient uptake. This is supported by stoichiometric modeling of E. coli metabolism, which illustrates the ATP requirement in E. coli for xylose transport to be a key limitation to xylitol production. Accordingly, a less energy-intensive xylose uptake mechanism is needed for further metabolic optimization of E. coli for xylitol production.

Thus, a need still exists for systems and methods that enable the economical production of xylitol in microbial systems from readily available substrates. In particular, a need exists for engineered E. coli strains that can serve as biocatalysts for the efficient conversion of biomass-derived sugars (such as glucose and xylose) into value-added products, such as xylitol.

### BRIEF SUMMARY OF THE INVENTION

The present invention provides novel microorganisms and methods for the production of xylitol from inexpensive raw products (preferably xylose, or xylose and glucose, mediums). The present invention also provides methods for constructing such microorganisms.

According to the subject invention, microbes are constructed to express several different xylose reductase (also referred to herein as XR) and xylitol dehydrogenase (also referred to herein XDH) enzymes to produce xylitol from xylose (or xylulose) in vivo. In a preferred embodiment, E coli are constructed to express XDH and/or XR to produce xylitol from a substrate consisting essentially of xylose (or xylulose). Certain embodiments of the invention provide engineered microbes capable of deriving reducing equivalents from carbon substrates (such as glucose) for the subsequent reduction of xylose or xylulose to xylitol.

In another embodiment, the invention uses E. coli as a biocatalyst for the conversion of xylose (or xylose in combination with other carbon substrates such as glucose) to xylitol. The present invention modifies the metabolism of E. coli by introducing and expressing various genes that improve the efficiency of xylose uptake. The modified E. coli of the invention enable a less energy-intensive uptake mechanism (than the one normally encountered with E. coli) for the production of xylitol via xylose reduction.

Another embodiment of the invention involves the use of E. coli as a host for heterologous, NAD(P)H-dependent transformations, whereby central metabolism serves as the cofactor regeneration system. Simple sugars can therefore serve as inexpensive energy sources (“cosubstrates”) to drive these transformations. Because glucose and xylose mixtures are often encountered during biomass hydrolysis, the engineered strains of the invention are advantageous in that they are capable of simultaneous glucose metabolism and xylitol production. The engineered microbes of the invention are particularly advantageous as a result of their ability to efficiently produce sugar alcohols and/or to catalyze other heterologous NAD(P)H-dependent transformations utilizing glucose as the energy source for cofactor regeneration. An efficient xylitol-producing strain of the invention can be used to “refine” biomass waste streams containing hexose and pentose sugars into xylitol.

In accordance with one embodiment of the subject invention, heterologous genes encoding enzymes, such as those encoding xylose reductase and xylitol dehydrogenase enzymes, are introduced to E. coli so that the transformed microorganism produces xylitol from xylose (or xylose in combination with a carbon substrate such as glucose). Such recombinant E. coli of the invention are preferably modified so that xylitol is stably produced with high yield when grown on a medium of xylose or a medium comprising xylose and carbon substrates (such as glucose).

When presented with a mixture of xylose in combination with carbon substrates such as glucose, native E. coli preferentially take up and metabolize the carbon substrates (in this example glucose) before the xylose. This phenomena is commonly referred to as diauxic growth. In one embodiment of the subject invention, diauxic growth is eliminated by replacing the native E. coli crp gene with a mutant form of the crp gene encoding a cAMP-independent CRP (also referred to herein as CRP* or CRP-in).

According to the present invention, the crp* gene is used to engineer E. coli that can produce xylitol from xylose (or xylulose) when in the presence of a carbon substrate (such as glucose). CRP* does not require cAMP to activate transcription of crp-controlled genes (for example, those genes responsible for xylose uptake and metabolism) and, therefore, the presence of CRP* appears to facilitate transcription of such genes even in the presence of carbon substrates such as glucose. Thus, without being bound to any theory, the crp* gene is particularly useful when a carbon substrate is present because it appears to enable less energy intensive uptake of xylose in the production of xylitol. Accordingly, using such transformed E. coli, the subject invention provides novel methods for the production of xylitol from substrates comprising a mixture of xylose and raw products. To ensure xylitol synthesis using such engineered E. coli, the microbes express reductase and/or dehydrogenase necessary for the synthesis of xylitol.

In a related embodiment of the invention, the native E. coli crp gene is replaced with crp* and the gene that encodes xylulokinase (referred to herein as the xylB gene) is deleted from the E. coli strain (an example of such a microorganism is referred to herein as the “PC09” strain). Such recombinant E. coli of the invention are capable of producing xylitol from xylose when in the presence of a carbon substrate such as glucose. To ensure xylitol synthesis using such engineered E. coli, the microbes express reductase and/or dehydrogenase necessary for the synthesis of xylitol. With this embodiment, glucose, as opposed to xylose, is the sole energy source that fuels the production of xylitol.

In certain related embodiments, PC09 is further transformed to include polynucleotide sequences that encode the XR and/or XDH enzymes to enable xylitol production. In other embodiments of the invention, polynucleotide sequences that encode the expression of XR and/or XDH enzymes are integrated into native E. coli strains.

Using the metabolic engineering processes described herein, the yield of xylitol from xylose, or xylose and carbon substrate, mediums is improved. Further, the ratio of xylitol produced per xylose (and when available, carbon substrate) consumed is improved when using the recombinant microbes of the invention. The microbial processes of the invention allow for xylitol production under mild conditions, with higher product purities and reduced downstream processing/purification requirements due to the efficient and essentially complete consumption by the transformed microorganisms of all sugars present in the feed.

### BRIEF DESCRIPTION OF THE FIGURES

FIG. 1 is an illustration of xylose uptake and metabolism into the pentose phosphate pathway (PPP) in E. coli, and options for xylitol production via heterologous XDH or XR.

FIG. 2A is a graphical illustration of glucose and xylose consumption by various embodiments of the invention.

FIG. 2B is a graphical illustration of growth curves of various embodiments of the invention when grown on glucose or xylose.

FIG. 3 is a graphical illustration of various xylitol-producing enzymes tested in an embodiment of the invention.

FIG. 4 is a graphical illustration of the amount of xylitol produced by various embodiments of the invention when grown on either xylose medium or xylose and glucose medium.

FIG. 5 is a graphical illustration of fermentation profiles for various embodiments of the invention.

FIG. 6 is a graphical illustration of the performance of a specific embodiment of the invention.

FIG. 7 illustrates the effects of crp* and ΔxylB on xylose and glucose consumption and xylulose secretion. Results are given for 4-hour and 8-hour time points from 50-ml shake flask cultures grown at 37° C. and containing LB medium supplemented with 100 mM each of glucose, xylose and MOPS buffer. PC05 and PC09 express the mutant CRP* protein; the xylB gene is deleted in PC07 and PC09. Data presented as the average of three values.

### BRIEF DESCRIPTION OF THE SEQUENCES

SEQ ID NO:1 is an oligonucleotide for amplifying the gene encoding xylitol dehydrogenase.

SEQ ID NO:2 is an oligonucleotide for amplifying the gene encoding xylitol dehydrogenase.

SEQ ID NO:3 is an oligonucleotide consistently included upstream of the start codon in certain embodiments of the invention.

### DETAILED DESCRIPTION OF THE INVENTION

The present invention provides novel microorganisms and methods for the production of xylitol in high yield from inexpensive raw products (such as sugars). The present invention also provides methods for constructing such microorganisms.

According to the subject invention, microbes are engineered that produce bulk amounts of xylitol, wherein the engineered microbes express at least one reductase and/or dehydrogenase during the synthesis of xylitol. In certain embodiments, microbes are engineered to express several different xylose reductase (also referred to herein as XR) and xylitol dehydrogenase (also referred to herein XDH) enzymes to produce xylitol from xylose (or xylulose) in vivo. In a preferred embodiment, E. coli are constructed to express XDH and/or XR to produce xylitol from a substrate consisting essentially of xylose (or xylulose). Certain embodiments of the invention provide engineered microbes capable of deriving reducing equivalents from carbon substrates (such as glucose) for the subsequent reduction of xylose or xylulose to xylitol.

As used herein, the terms gene and polynucleotide sequence are used interchangeably. With these terms, the present invention encompasses nucleotide sequences that encode for or correspond to a particular sequence of nucleic acids (such as ribonucleic acids) or amino acids that comprise all or part of one or more products (such as polypeptides, proteins, or enzymes), and may or may not include regulatory sequences, such as promoter sequences, which determine, for example, the conditions under which the gene is expressed.

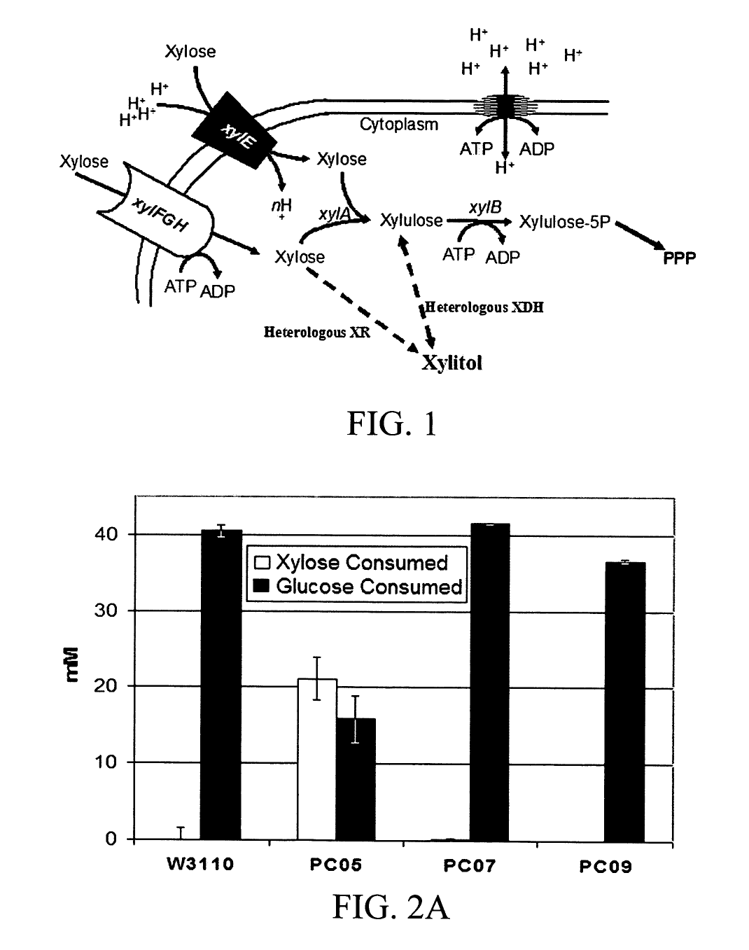

As shown in FIG. 1, xylose uptake in naturally-occurring E. coli occurs primarily through either a high-affinity ATP-binding cassette (ABC) transporter (such as the xylose ABC transporter XylFGH) or a low-affinity proton symporter (such as the D-xylose proton symporter XylE) (Neidhardt F C et al., Escherichia coli and Salmonella: Cellular and Molecular Biology, Washington D.C.: ASM Press; 1996), although studies suggest that XylE has very low activity even under high xylose concentrations (50 mM) (Hasona A et al., “Pyruvate formate lyase and acetate kinase are essential for anaerobic growth of Escherichia coli on xylose,” J Bacter, 186:7593-7600 (2004)). Xylose is metabolized by first being isomerized to xylulose by xylulose isomerase (XylA) followed by xylulokinase (XylB)-catalyzed xylulose phosphorylation to xylulose-5-phosphate, which then enters the pentose phosphate (PP) pathway. E. coli cannot naturally synthesize (or metabolize) xylitol. Xylitol production is possible by either expression of xylose reductase for direct reduction of xylose, or expression of the reversible xylitol dehydrogenase, whereby xylulose is reduced to xylitol (see FIG. 1).

E. coli exhibit diauxic growth characteristics such that glucose is preferentially assimilated before xylose as a result of cAMP-dependent transcriptional activation of genes required for xylose uptake and metabolism (Hernandez-Montalvo V et al., “Characterization of sugar mixtures utilization by an Escherichia coli mutant devoid of the phosphotransferase system,” Applied Microbiology Biotech, 57:186-191 (2001); Song S G and C Park, “Organization and regulation of the D-xylose operons in Escherichia coli K-12: XylR acts as a transcriptional activator,” J Bacteriology, 179:7025-7032 (1997)). According to the subject invention, microbes are engineered to constitutively uptake xylose in the production of xylitol.

In certain embodiments of the invention, a method is provided for using biotransformed E. coli to produce xylitol from sources comprising xylose alone or in combination with other carbon substrates (such as glucose). In one embodiment, E. coli are engineered, which constitutively uptake xylose due to the replacement of the native crp gene with a mutant gene (whose mutations correspond to three amino acid substitutions) encoding a cAMP-independent CRP variant (denoted “CRP*” or “CRP-in”). Methods for isolating crp* genes are well-known to the skilled artisan. See, for example, methods disclosed by Eppler T and W Boos, “Glycerol-3-phosphate-mediated repression of malT in Escherichia coli does not require metabolism, depends on enzyme IIA(Glc) and is mediated by cAMP levels,” Molecular Microbiology, 33:1221-1231 (1999). Such engineered E. coli, which express CRP* (also referred to herein as PC05), are able to take up xylose in the presence of glucose during xylitol production. Preferably, such E. coli express a reductase and/or dehydrogenase necessary for the synthesis of xylitol. Accordingly, the subject invention provides advantageous methods for the bioproduction of xylitol from xylose-based sources using the PC05 strain of the invention.

The E. coli strains of the invention are particularly useful for the conversion of sugar mixtures comprising xylose into value-added products (such as xylitol). In addition to allowing for transcription of xylose transporter genes (and/or genes that code for transporters capable of allowing xylose uptake), the crp* genotype should promote activation of other CRP-controlled genes involved in tricarboxylic acid cycle (TCA) function, such as citrate synthase (gltA), succinate dehydrogenase (sdh), and α-ketoglutarate dehydrogenase (suc) (Neidhardt F C et al., Escherichia coli and Salmonella: Cellular and Molecular Biology, Washington D.C.: ASM Press; 1996), thereby increasing cofactor availability to drive xylitol production.

In other related embodiments, the PC05 strain is further engineered to additionally contain a deletion in the xylB gene encoding xylulokinase (ΔxylB), which prevents metabolism of xylose while still allowing conversion of xylose to xylulose for XDH-driven xylitol production (this strain is also referred to herein as PC09). The xylB gene deletion simplifies monitoring of carbon trafficking and NAD(P)H regeneration in future metabolic engineering and analyses in strains derived from PC09. Specifically, deletion of the xylB gene prevents xylose metabolism so that the carbon substrate is the sole source of fuel used in the production of xylitol. In a preferred embodiment, E. coli engineered with crp* gene as well as a deletion in the xylB gene are presented to a medium comprising xylose and glucose, wherein the deletion of the xylB gene enables the glucose to fuel the reaction of reducing the reserved xylose to xylitol while the crp* allows for xylose uptake in the presence of glucose. Preferably, such E. coli express a reductase and/or dehydrogenase necessary for the synthesis of xylitol.

In other related embodiments, PC09 is further transformed by introducing polynucleotides that encode XR and/or XDH enzymes. When functional XR and/or XDH enzymes are expressed in PC09 while the strain is grown on a glucose-plus-xylose mixture in a defined salt medium, glucose is metabolized with the concomitant reduction of xylose (or xyluylose) to xylitol. That is, a fraction of the reduced cofactors derived from glucose assimilation are oxidized via the XR- and/or XDH-catalyzed reduction of xylose or xylulose. As a result, glucose-xylose mixtures can be efficiently converted into xylitol using the PC09 E. coli strain of the invention.

In yet another embodiment of the invention, native E. coli strains are transformed to express functional XR and/or XDH enzymes. Such engineered microbes are capable of xylose metabolism for use in xylitol production (for example, conversion of xylose to xylitol, where xylose serves as the sole sugar source for xylitol production).

The skilled artisan would readily understand the many methods available for integrating and/or removing genes in a microbe such as E. coli. For example, the skilled artisan would recognize that P1 phage transduction can be used to integrate mutant crp* (or genes that encode the expression of XR and/or XDH enzymes) into E. coli. Other available methods for integrating genes into E. coli include, but are not limited to, transposon (Berg, D. E. and Berg, C. M., Bio/Tecnol., 1, 417 (1983)), Mu phage (Japanese Patent No. 2-109985), or homologous recombination (Experiments in Molecular Genetics, Cold Spring Harbor Lab. (1972)).

According to the subject invention, the skilled artisan would readily recognize those microorganisms that may be used as the source of the gene encoding xylulokines, XR, and/or XDH. Examples of such microorganisms include, for example, Gluconobacter cerinus, Gluconobacter oxydans, Acetobacter aceti, Acetobacter liquefaciens, Acetobactor pasteurianus, Frateuria aurantia, Bacillus subtilis, Bacillus megaterium Proteus rettgeri, Serratia marcescens, Corynebacterium callunae, Brevibacterium ammoniagenes, Flavobacterium aurantinum, Flavobaterium rhenanum Pseudomonas badiofaciens, Pseudomonas chlororaphis, Pseudomonas iners, Rhodococcus rhodochrous, Achromobacter viscosus, Agrobacterium tumefaciens, Agrobacterium radiobacter, Arthrobacter paraffineus, Arthrobacter hydrocarboglutamicas, Azotobacter indicus, Brevibacterium ketoglutamicum, C. boidinii, Corynebacterium faciens, Erwinia amylovora, Flavobacterium peregrinum, Flavobacterium fucatum, Micrococcus sp. CCM825, Nocardia opaca, Planococcus eucinatus, Pseudomonas synxantha, Rhodococcus erythropolis, Morganella morganii, Actinomadura madurae, Actinomyces violaceochromogenes, Streptomyces coelicolor, Streptomyces flavelus, Streptomyces griseoulus, Streptomyces lividans, Streptomyces olivaceus, Streptomyces tanashiensis, Streptomyces virginiae, Streptomyces antibioticus, Streptomyces cacaoi, Streptomyces lavendulae, Pichia stipitis and so forth.

Among the aforementioned microorganisms, the nucleotide sequences of the genes encoding xylitol dehydrogenase (XDH) derived from, for example, Pichia stipitis (FEBS Lett., 324, 9 (1993)) or Morganella morganii (DDBJ/GenBank/EMBL Accession No. L34345) have been reported, and therefore a gene encoding xylitol dehydrogenase can be obtained by synthesizing primers based on the nucleotide sequences of these genes encoding xylitol dehydrogenase, and performing polymerase chain reaction (PCR) using chromosomal DNA of microorganisms such as Morganella morganii ATCC 25829 as a template. Specific examples of the primers include the oligonucleotides for amplifying the gene encoding xylitol dehydrogenase of Morganella morganii, which have the nucleotide sequences represented in the accompanying Sequence Listing as SEQ ID NO:1 (DNA; artificial sequence; synthetic DNA: CGGGAATTCGATATCATTTT AATGAA) and SEQ ID NO:2 (DNA; artificial sequence; synthetic DNA: GGCGGATCCG CAGTCAATAC CGGCATAGA).

The vector used for introducing a polynucleotide sequence encoding xylose reductase and/or xylitol dehydrogenase into a host microorganism may be any vector so long as it can replicate in the host microorganism. Specific examples thereof include plasmid vectors such as pBR322, pTWV228, pMW119, pUC19 and pUC18.

In a specific embodiment, to prepare recombinant DNA containing the gene encoding xylose reductase and/or xylitol dehydrogenase, the gene(s) is/are ligated to a vector that functions in Escherichia bacteria. The vector can be digested with a restriction enzyme matching the terminal sequence of the gene encoding, for example, xylitol dehydrogenase, and the sequences can be ligated. The ligation is usually attained by using a ligase such as T4 DNA ligase.

The recombinant plasmid prepared as described above can be introduced into a host microorganism by a method reported for Escherichia coli such as a method of D. A. Morrison (Methods in Enzymology, 68, 326 (1979)) or a method in which recipient cells are treated with calcium chloride to increase permeability for DNA (Mandel, M. and Higa, A., J. Mol. Biol., 53, 159 (1970)). Alternatively, a gene encoding xylose reductase and/or xylitol dehydrogenase can also be incorporated into the chromosome of the host by a method utilizing transduction, transposon (Berg, D. E. and Berg, C. M., Bio/Tecnol., 1, 417 (1983)), Mu phage (Japanese Patent Laid-open [KOKAI] No. 2-109985), or homologous recombination (Experiments in Molecular Genetics, Cold Spring Harbor Lab. (1972)).

As the promoter for the expression of the gene(s) to be presented in the host microorganism (for example, gene encoding xylose reductase and/or xylitol dehydrogenase), when a promoter specific for the gene functions in host cells, this promoter can be used. Alternatively, it is also possible to ligate a foreign promoter to a DNA encoding the desired protein so as to obtain the expression under the control of the promoter. As such a promoter, when an Escherichia bacterium is used as the host, lac promoter, trp promoter, trc promoter, tac promoter, P_{R} promoter and P_{L} promoter of lambda phage, tet promoter, amyE promoter, spac promoter and so forth can be used. Further, it is also possible to use an expression vector containing a promoter like pUC19, and insert a DNA fragment, encoding for example xylitol dehydrogenase, into the vector so that the fragment can be expressed under the control of the promoter.

When a microorganism contains a gene (such as those encoding xylose reductase and/or xylitol dehydrogenase), xylitol production can be enhanced by replacing an expression regulatory sequence with a stronger one such as the promoter of the gene itself (see Japanese Patent No. 1-215280). For example, all of the aforementioned promoters functioning in Escherichia bacteria have been known as strong promoters. In a preferred embodiment, medium-copy (15-30 per cell) plasmid (pFLAG, containing a pBR322 origin) with a strong tac promoter is used to express XR enzymes in biotransformed microbes of the invention.

Methods for preparation of chromosome DNA, PCR, preparation of plasmid DNA, digestion and ligation of DNA, transformation, design and synthesis of oligonucleotides used as primers and so forth may be usual ones well known to those skilled in the art. Such methods are described in, for example, Sambrook, J., Fritsch, E. F., and Maniatis, T., “Molecular Cloning: A Laboratory Manual, Second Edition,” Cold Spring Harbor Laboratory Press (1989) and so forth.

Advantageous methods for producing xylitol according to the subject invention comprise the following steps: growing a transformed microbe of the invention on a carbon substrate and xylose medium and collecting the xylitol produced by the microbe. The transformed microbes that can be used to produce xylitol include, but are not limited to, E. coli that have integrated the crp* gene and that have had the xylB gene deleted; and crp* E. coli that have been transformed to include genes that encode XR and/or XDH enzymes.

In one embodiment, the invention uses biotransformed E. coli as a biocatalyst for the conversion of xylose to xylitol. Preferably, the biotransformed E. coli produce xylitol when grown on mediums that include xylose. More preferably, the subject microorganisms produce xylitol when grown on a xylose medium, or a medium of xylose in combination with another carbon substrate.

The medium used for the culture of the subject microorganisms may be a usual medium that contains a carbon substrate, a nitrogen source, inorganic ions suitable for the microorganism, as well as other organic components, if necessary. Carbon substrates for use in accordance with the invention include carbohydrates (including monosaccharides, disaccharides, trisaccharides, and polysaccharides). Examples of carbon substrates that can be used in accordance with the invention include, but are not limited to, starches (such as corn, oat, wheat, rye, rice, barley, millet, quinoa, potato, and the like); sugars (such as glucose, fructose, mannose, galactose, maltose, lactose, cellobiose, gentiobiose, melibiose, sucrose, trehalose, manitriose, rabinose, rhamnose, raffinose); starch hydrolysate; alcohols (such as glycerol or sorbitol); or organic acids (such as fumaric acid, citric acid, or succinic acid). The transformed microbes of the invention are preferably grown on mediums of xylose alone or in combination with other sugars, most preferably glucose.

As the nitrogen source, inorganic ammonium salts such as ammonium sulfate, ammonium chloride, or ammonium phosphate; organic nitrogen such as soybean hydrolysate; ammonia gas; or aqueous ammonia can be used.

Cultivation of the transformed microorganisms of the invention can be carried out under aerobic condition for about 16-120 hours. The cultivation temperature is preferably controlled at about 25° C. to 45° C., and pH is preferably controlled at about 5-8 during cultivation. Inorganic or organic, acidic, or alkaline substances as well as ammonia gas or the like can be used for pH adjustment.

As discussed above, the microorganism of the present invention can be obtained by transforming a bacterium to express certain enzymes useful in the production of xylitol from xylose and/or glucose. Examples of bacterium that can be used in the present invention include, but are not limited to, Escherichia coli, Bluconobacter oxydans, Bluconobacter asaii, Achromobacter delmarvae, Achromobacter viscosus, Achromobacter lacticum, Agrobacterium tumefaciens, Agrobacterium radiobacter, Alcaligenes faecalis, Arthrobacter citreus, Arthrobacter tumescens, Arthrobacter paraffineus, Arthrobacter hydrocarboglutamicus, Arthrobacter oxydans, Aureobacterium saperdae, Azotobacter indicus, Brevibacterium ammoniagenes, Brevibacterium divaricatum, Brevibacterium lactofermentum, Brevibacterium flavum, Brevibacterium globosum, Brevibacterium fuscum, Brevibacterium ketoglutamicum, Brevibacterium helcolum, Brevibacterium pusillum, Brevibacterium testaceum, Brevibacterium roseum, Brevibacterium immariophilium, Brevibacterium linens, Brevibacterium protopharmiae, C. boidinii, Corynebacterium acetophilium, Corynebacterium glutamicum, Corynebacterium callunae, Corynebacterium acetoacidophilum, Corynebacterium acetoglutamicum, Enterobacter aerogenes, Erwinia amylovora, Erwinia carotovora, Erwinia herbicola, Erwinia chrysanthemi, Flavobacterium peregrinum, Flavobacterium fucatum, Flavobacterium aurantinum, Flavobacterium rhenanum, Flavobacterium Sewanense, Flavobacterium breve, Flavboacterium meningosepticum, Micrococcus sp. CCM825, Morganella morganii, Nocardia opace, Nocardia rugosa, Planococcus eucinatus, Proteus rettgeri, Propionibacterium shermanii, Pseudomonas synxantha, Pseudomonas azotoformans, Pseudomonas fluorescens, Pseudomonas ovalis, Pseudomonas stutzeri, Pseudomonas acidovolans, Pseudomonas mucidolens, Pseudomonas test6osteroni, Pseudomonas aeruginosa, Rhodococcus erythropolis, Rhodococcus rhodochrous, Rhodococcus sp. ATCC 15592, Rhodococcus sp. ATCC 19070, Sprorsarcina ureae, Staphylococcus aureus, Vibrio metschnikovii, Vibrio tyrogenes, Actinomadura madurae, Actinomyces violaceochromogenes, Kitasatosporia parulosa, Streptomyces coelicolor, Strptomyces flavelus, Streptomyces griseolus, Streptomyces lividans, Streptomyces olivaceus, Streptomyces tanashiensis, Streptomyces virginiae, Streptomyces antibioticus, Streptomyces cacaoi, Streptomyces lavendulae, Streptomyces viridochromogenes, Aeromonas salmonicida, Bacillus pumilus, Bacillus circulans, Bacillus thiaminolyticus, Escherichia freundii, Microbacterium ammoniaphilum, Serratia marcescens, Salmonella typhimurium, Salmonella schottmulleri, Xanthomonas citri and so forth.

Xylitol can be produced in a reaction mixture by contacting a culture containing transformed microorganisms prepared in accordance with the present invention with xylose and/or glucose. In other embodiments, “resting cells” or cells that are still metabolically active but deprived of a nutrient such as nitrogen or phosphorous can be transformed and used in accordance with the subject invention. The resting cells of the invention can further include a protein synthesis inhibitor so that energy sources (such as glucose or other sugar) are converted into reducing equivalents (such NAD(P)H cofactors) to drive the reduction of xylose or xylulose to produce xylitol. With such resting cells, the energy source is not “wasted” for ensuring more cell mass, rather, the energy from metabolizing the energy source will be available for xylitol production.

Following are examples that illustrate procedures for practicing the invention. These examples should not be construed as limiting. All percentages are by weight and all solvent mixture proportions are by volume unless otherwise noted.

### EXAMPLE 1—MATERIALS AND METHODS

E. coli W3110 (ATCC 27325) and derivative strains were maintained on plates containing either Luria-Bertani (LB) medium or minimal medium containing mineral salts (per liter: 3.5 g KH₂PO₄; 5.0 g K₂HPO₄; 3.5 g (NH₄)₂HPO₄, 0.25 g MgSO₄. 7 H₂O, 15 mg CaCl₂. 2 H₂O, 0.5 mg thiamine, and 1 ml of trace metal stock), glucose (2%), and 1.5% agar. The trace metal stock was prepared as described by Causey T B et al., “Engineering the metabolism of Escherichia coli W3110 for the conversion of sugar to redox-neutral and oxidized products: Homoacetate production,” Proceedings of the National Academy of Sciences of the United States of America, 100:825-832 (2003). 4-Morpholinopropanesulfonic acid (MOPS) was added to liquid media for pH control (50 mM, pH 7.0), but was not included in medium used for 10-L fermentations. Antibiotics were included as appropriate (kanamycin, 50 mg L⁻¹; ampicillin, 50 mg L⁻¹; apramycin, 50 mg L⁻¹ and tetracycline, 12.5 mg L⁻¹) and β-D-thiogalactopyranoside (IPTG) (100 μM) was to induce protein production under tac promoter control.

Standard methods were used for plasmid construction, phage P1 transduction, electroporation, and polymerase chain reaction (PCR) (see Miller J H, A short course in bacterial genetics: A laboratory manual and handbook for Escherichia coli and related bacteria, Cold Spring Harbor, N.Y.: Cold Spring Harbor Press (1992); and Sambrook J and Russell D W, Molecular cloning: A laboratory manual, Cold Spring Harbor, N.Y.: Cold Spring Harbor Press (2001)). Strains used in this study are listed in Table 1 (see Example 2 below).

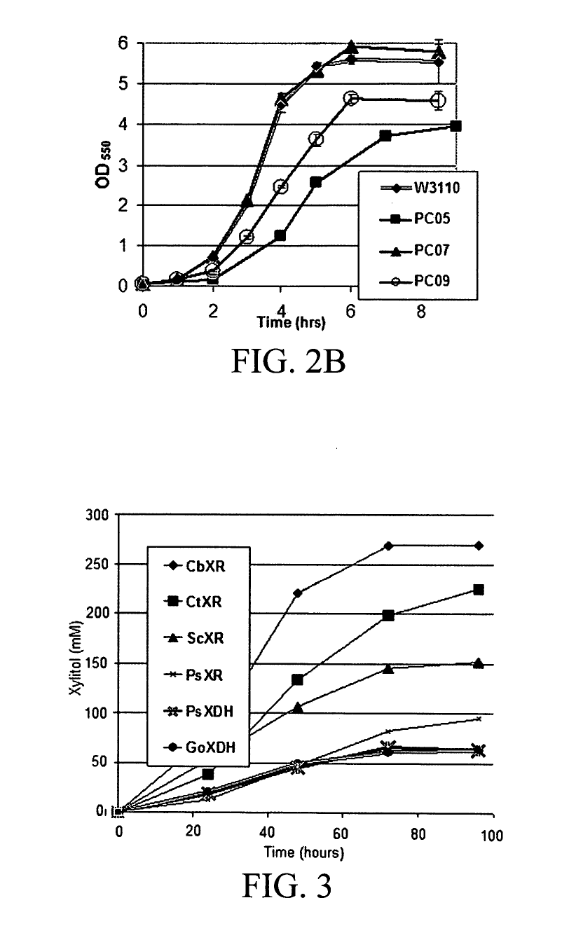

E. coli K-12 strain W3110 (ATCC 27325) was used as the wild-type strain. Strain ET25, carrying the crp* gene next to a Tn::10 marker was obtained from Dr. W. Boos (University of Konstanz, Konstanz, Germany; Eppler T and Boos W, “Glycerol-3-phosphate-mediated repression of malT in Escherichia coli does not require metabolism, depends on enzyme IIA(Glc) and is mediated by cAMP levels,” Mol Microbiol, 33:1221-1231 (1999)). The crp* gene was introduced into W3110 via P1 phage transduction using a lysate from strain ET25 followed by selection on tetracycline plates. The resulting strain was named PC05. The crp* phenotype was verified in two ways. First, several Tet^{R} transductants were grown in LB broth containing glucose (1%) and xylose (1%). Mid log-phase cells were harvested and washed several times in phosphate buffer containing kanamycin (100 ug/ml). After allowing sufficient time for residual sugars to be cleared, the cells were resuspended a final time in buffer containing xylose (1%), kanamycin, and 1% triphenyltetrazolium chloride (TTC). Reduction of TTC results in red color formation and indicates constitutive xylose utilization. The crp* phenotype was additionally confirmed using HPLC to verify simultaneous glucose and xylose consumption in batch cultures, as described in the text and shown in FIG. 2.

Disruption of the xylB gene was accomplished using previously described methods (see, for example, Causey T B et al., “Engineering the metabolism of Escherichia coli W3110 for the conversion of sugar to redox-neutral and oxidized products: Homoacetate production,” Proceedings of the National Academy of Sciences of the United States of America, 100:825-832 (2003); Datsenko K A and Wanner B L, “One-step inactivation of chromosomal genes in Escherichia coli K-12 using PCR products,” Proceedings of the National Academy of Sciences of the United States of America, 97:6640-6645 (2000); Martinez-Morales F et al., “Chromosomal integration of heterologous DNA in Escherichia coli with precise removal of markers and replicons used during construction,” J Bacteriology, 181:7143-7148 (1999); and Posfai G et al., “Versatile insertion plasmids for targeted genome manipulations in bacteria: Isolation, deletion, and rescue of the pathogenicity island LEE of the Escherichia coli O157:H7 genome,” J Bacteriology, 179:4426-4428 (1997)).

Briefly, the xylB gene was amplified from W3110 genomic DNA using Taq DNA polymerase (New England Biolabs, Ipswich, Mass.) and xylB ORFmers (Sigma-Genosys, Woodlands, Tex.) as primers. The resulting PCR fragment was “TOPO-cloned” into vector pCR2.1-TOPO (Invitrogen, Carlsbad, Calif.). A 240-bp fragment within the xylB gene was removed by digestion with BsiWI followed by Klenow fill-in. Next, a 1956-bp SmaI fragment containing an apramycin resistance gene (aac) flanked by FRT flipase recognition sequences (isolated from lab plasmid pLOI3421) was ligated in place of the deleted xylB fragment. The corresponding ligation product (plasmid pLOI3807) therefore contains the sequence xylB′-FRT-aac-FRT-xylB″. This sequence was PCR-amplified using the xylB ORFmers as primers, and the PCR product was electroporated into E. coli W3110 expressing Red recombinase from plasmid pKD46 (Datsenko K A, and Wanner B L, “One-step inactivation of chromosomal genes in Escherichia coli K-12 using PCR products,” Proceedings of the National Academy of Sciences of the United States of America, 97:6640-6645 (2000)). Apramycin-resistant colonies arising from homologous recombination of the xylB deletion construct were selected and verified by PCR.

Strain PC06 (W3110, ΔxylB::FRT-aac-FRT) was used for moving the xylB deletion into other strains (e.g., PC05) by P1 phage transduction. FRT-flanked antibiotic resistance was deleted using a temperature-conditional plasmid (pFT-A) expressing FLP recombinase from a chlortetracycline-inducible promoter (Posfai G et al., “Versatile insertion plasmids for targeted genome manipulations in bacteria: Isolation, deletion, and rescue of the pathogenicity island LEE of the Escherichia coli O157:H7 genome,” J Bacteriology, 179:4426-4428 (1997)).

All reductase and dehydrogenase genes were amplified using high-fidelity polymerases (Stratagene, La Jolla, Calif. (Pfu) and Invitrogen, Carlsbad, Calif. (Pfx)) and primers containing appropriate restriction sites for ligation into a multiple cloning site directly downstream of a tac promoter and upstream of a transcription termination sequence in plasmid pLOI3809. The set of unique restriction sites used for each forward and reverse primer depended on the restriction sites found within the corresponding gene sequence being cloned. All genes were cloned to contain the same sequence upstream of the start codon: AGGAGGACAGCTatg . . . (SEQ ID NO: 3; Shine Delgamo sequence is underlined).

The source of DNA used to amplify each gene were as follows: GoXDH: genomic DNA prep of Gluconobacter oxydans (ATCC 621); GRE3: genomic prep of Baker's yeast; CtXR: genomic DNA prep of Candida tenuis (CBS 4435); PsXDH and PsXR: plasmids pCR2.1-TOPO-PSXYL2 and pCR2.1-TOPO-PSXYL2, respectively, which were provided by Dr. T. W. Jeffries (Kang M H et al., “Molecular characterization of a gene for aldose reductase (CbXYL1) from Candida boidinii and its expression in Saccharomyces cerevisiae,” Appl Biochemistry Biotech, 105:265-276 (2003)); CbXR: plasmid pRS424-CBAR, also provided by Dr. T. W. Jeffries. The cloned genes were sequenced to verify fidelity. One discrepancy was noted in the amino acid sequence of GoXDH compared to that reported (Sugiyama M et al., “Cloning of the xylitol dehydrogenase gene from Gluconobacter oxydans and improved production of xylitol from D-arabitol,” Bioscience Biotechn Biochem, 67:584-591 (2003)): Thr159 rather than Ala159 (this was verified by sequencing products from two separate PCR reactions). In addition, an error in primer synthesis resulted in a point mutation (Thr6Ala) in the ScXR (GRE3) amino acid sequence.

Strain JC12, which contains the CbXR gene under control of a tac promoter integrated into the PC09 chromosome at the attHK022 site, was constructed using a CRIM plasmid as described by Haldimann and Wanner (“Conditional-replication, integration, excision, and retrieval plasmid-host systems for gene structure-function studies of bacteria,” J Bacteriology, 183:6384-6393 (2001)). Briefly, a ˜4313-bp FspI fragment containing the lacI gene and the CbXR gene downstream of a tac promoter and upstream of a transcriptional terminator, was excised from plasmid pLOI3815. This fragment was ligated into the SmaI site of plasmid pPCC20. (Plasmid pPCC20 was constructed from CRIM plasmid pAH68 (Haldimann A et al., “Conditional-replication, integration, excision, and retrieval plasmid-host systems for gene structure-function studies of bacteria,” J Bacteriology, 183:6384-6393 (2001)) by replacing the bla gene (digested with MslI), with a 1956-bp SmaI fragment (containing the FRT-aac-FRT sequence, described above) from plasmid pLOI3421.) The resulting CRIM plasmid pPCC100, containing the Ptac-controlled CbXR gene and FRT-flanked apramycin resistance, was integrated into the PC09 chromosomal attHK022 site using helper plasmid pAH69. Single-copy integrants were selected on apramycin plates and confirmed by PCR as described by Haldimann et al. Apramycin resistance was removed by FLP-mediated recombination using plasmid pFT-A (Posfai G et al., “Versatile insertion plasmids for targeted genome manipulations in bacteria: Isolation, deletion, and rescue of the pathogenicity island LEE of the Escherichia coli O157:H7 genome,” J Bacteriology, 179:4426-4428 (1997)) and products were verified by PCR, resulting in strain JC12.

Shake Flask Cultures

All cultures were performed in duplicate or triplicate, and all data points reported are the average of at least two experiments, except for the single, 10-liter fermentation. Shake flasks cultures for xylitol production contained 50 mL medium in 250-mL baffled flasks and were grown at 30° C. and 250 rpm. Cultures with strains not harboring plasmids for determining simultaneous xylose and glucose utilization (FIGS. 2A and 2B) contained 50 mL medium in 250-mL baffled flasks and were grown at 37° C., 250 rpm.

All LB and minimal medium cultures were inoculated to an initial OD₅₅₀ of 0.1 from seed cultures of the same medium. Seed cultures were prepared by inoculating colonies from a fresh plate (LB plates for LB cultures, minimal medium plates containing 2% glucose for minimal medium cultures) into 3 ml of medium (13×100 mm tube) containing 50 mM MOPS. Seeds were grown to an OD₅₅₀ of 2.0-4.0, and shake flask cultures were inoculated directly from the seed cultures by dilution to a final OD₅₅₀ of 0.1. Kanamycin was included in all plasmid-containing cultures, and protein expression was induced with 100 μM IPTG in shake flasks at the time of inoculation.

Fermentation

The fermentation described herein was accomplished using a New Brunswick Bioflow 3000 fermentor with a 10 L working volume (30° C., dual Rushton impellers, 450 rpm). The fermentation seed culture was prepared as follows: colonies growing on a fresh minimal plate (2% glucose) were used to inoculate four 3-mL pre-seed cultures (13×100 mm tubes) containing 100 mM glucose and 50 mM MOPS. Pre-seeds were grown at 37° C. to OD550 of about 2.5, and were used to inoculate (by dilution) three 200-ml seed cultures in 1-L shake flasks grown at 30° C., 250 rpm. These seeds were harvested (5000×g, 25° C.) when the OD₅₅₀ reached about 1.8 and cells were resuspended in 100-mL spent medium to provide an inoculum of 33 mg dry cell weight L⁻¹ in the 10-L working volume (OD₅₅₀ about 0.1).

Resting Cells

Cells used for resting cell experiments were first grown in 200-mL shake flask cultures (in 1-L flasks) in minimal medium containing 100 mM glucose and 50 mM xylose under inducing conditions (100 μM IPTG). During rapid growth, when the OD₅₅₀ was 2.0-4.0, cells were harvested and resuspended to a “working density” of 2.0 in 30-mL minimal media containing 50 mM glucose and 300 mM xylose, but lacking a nitrogen source (ammonia). Maximum resting cell productivity occurred when cells were shaken at 30° C., 250 rpm in baffled flasks. Cell densities did not change significantly after resuspension for as many as four days, and addition of chloramphenicol had no effect on resting cell results. Residual concentrations of xylitol produced in control experiments in which glucose was not added to the resting cells was subtracted from the xylitol produced in the presence of glucose and xylose when calculating the yield on (reduced) product per glucose consumed (also referred to herein as the Y_{RPG}).

Analyses

Xylitol, xylose, xylulose, glucose and organic acid concentrations were determined using a Shimadzu LC-10AD HPLC equipped with a UV monitor (210 nm) and refractive index detector. Products were separated using a Bio-Rad HPX-87H column (10 μl injection) with 4 mM H₂SO₄ as the mobile phase (0.4 ml min⁻¹, 45° C.). Cell mass was estimated by measuring OD₅₅₀ nm (1.0 OD_{550 nm} is equivalent to 0.33 g L-⁻¹ dry cell weight).

### EXAMPLE 2—CHARACTERIZATION OF STRAINS

Table 1 lists the strains and plasmids used in accordance with the Examples of the subject invention. Our initial studies involved expression of XDH cloned from Gluconobacter oxydans (Sugiyama et al. 2003) in E. coli W3110 and its derivative PC07, containing a xylB (xylulokinase gene) deletion. Low concentrations of xylitol were produced in LB broth containing xylose alone, xylose plus sorbitol, and xylose plus glucose. Neither strain produced xylitol in the absence of XDH expression. For further studies we developed strain PC05, constitutive in xylose metabolism due to the replacement of the native crp gene with a mutant gene (corresponding to three amino acid substitutions) encoding a cAMP-independent CRP variant (denoted “CRP*” or “CRP-in”) (Eppler and Boos 1999). The CRP* phenotype should promote xylose uptake in the presence of glucose by activating the native xylose transporters and/or by activating other CRP-controlled promiscuous transporters capable of xylose uptake (particularly when xylose concentrations are high (>50 mM)). To prevent assimilation of xylose carbon into metabolism but still allow for constitutive xylose uptake and conversion to xylulose (for the case of XDH-driven xylitol production), the xylB gene was deleted from strain PC05, resulting in strain PC09.

TABLE 1
Strains and plasmids

| Strains/Plasmids | Relevant Characteristics | Reference |
|---|---|---|
| Strains | | |
| W3110 | wild type | ATCC 27325 |
| ET25 | E. coli K-12, crp*::Tn10 (Tet^{R}) | Eppler et al. |
| PC05 | W3110, crp*::Tn10 (Tet^{R}) | Subject |
| | | Invention |
| PC06 | W3110, ΔxylB::FRT-aac-FRT | Subject |
| | (Apr^{R}) | Invention |
| PC07 | W3110, ΔxylB::FRT | Subject |
| | | Invention |
| PC08 | PC05, ΔxylB::FRT-aac-FRT | Subject |
| | (Tet^{R}, Apr^{R}) | Invention |
| PC09 | PC05, ΔxylB::FRT (Tet^{R}) | Subject |
| | | Invention |
| JC12 | PC09, CbXR integrated at | Subject |
| | attHK022 site | Invention |
| Plasmids | | |
| pLOI3809 | kan, pBR322-origin vector for | Subject |
| | expression of XR or XDH, under | Invention |
| | control of tac promoter | |
| pLOI3815 | pLOI3809 carrying CbXR gene | Subject |
| | | Invention |
| pPCC04 | pLOI3809 carrying ScXR gene | Subject |
| | | Invention |
| pPCC05 | pLOI3809 carrying CtXR gene | Subject |
| | | Invention |
| pPCC06 | pLOI3809 carrying PsXDH gene | Subject |
| | | Invention |
| pPCC07 | pLOI3809 carrying PsXR gene | Subject |
| | | Invention |
| pPCC12 | pLOI3809 carrying GoXDH gene | Subject |
| | | Invention |
| W3110 | wild type | ATCC 27325 |
| ET25 | E. coli K-12, crp*::Tn10 (Tet^{R}) | |
| PC05 | W3110, crp*::Tn10 (Tet^{R}) | Subject |
| | | Invention |
| PC06 | W3110, ΔxylB::FRT-aac-FRT | Subject |
| | (Apr^{R}) | Invention |
| PC07 | W3110, ΔxylB::FRT | Subject |
| | | Invention |
| PC08 | PC05, ΔxylB::FRT-aac-FRT | Subject |
| | (Tet^{R}, Apr^{R}) | Invention |
| pCR2.1-TOPO | bla kan, TOPO ™ TA cloning | Invitrogen |
| | vector | |
| pFT-A | bla flp low-copy vector | |
| | containing recombinase and | |
| | temperature-conditional pSC101 | |
| | replicon | |
| pKD46 | bla γ β exo low-copy | |
| | vector containing red | |
| | recombinase and temperature- | |
| | conditional pSC101 replicon | |
| pLOI3421 | bla, SmaI fragment with FRT | Subject |
| | flanked aac gene | Invention |
| pLOI3806 | bla kan xylB | Subject |
| | | Invention |
| pLOI3807 | bla kan xylB::FRT-aac-FRT | Subject |
| | | Invention |
| pLOI3809 | kan, pBR322-origin vector for | Subject |
| | expression of XR or XDH, under | Invention |
| | control of tac promoter | |
| pLOI3815 | pLOI3809 carrying CbXR gene | Subject |
| | | Invention |
| pPCC04 | pLOI3809 carrying ScXR gene | Subject |
| | | Invention |
| pPCC05 | pLOI3809 carrying CtXR gene | Subject |
| | | Invention |
| pPCC06 | pLOI3809 carrying PsXDH gene | Subject |
| | | Invention |
| pPCC07 | pLOI3809 carrying PsXR gene | Subject |
| | | Invention |
| pPCC12 | pLOI3809 carrying GoXDH gene | Subject |
| | | Invention |

Tet = tetracycline,
Apr = apramycin,
Kan = kanamycin,
Amp = ampicillin

To verify elimination of diauxic growth by strains carrying the crp* gene, W3110, PC05, PC07, and PC09 were grown in glucose-xylose mixtures and sugar concentrations were monitored during growth. FIG. 2A shows the concentrations of xylose and glucose consumed after 10 hours of growth in batch cultures containing Luria-Bertani (LB) medium supplemented with 100 mM each of glucose and D-xylose (initially) and 100 mM 4-morpholinopropane-sulfonic acid (MOPS) buffer. Whereas xylose uptake in the presence of glucose is negligible for strains W3110 and PC07, PC05 (crp*) metabolizes both sugars simultaneously. Deletion of xylB gene from PC05 results in PC09, which is unable to metabolize xylose. Similar results were obtained when these strains were grown in minimal medium containing glucose and xylose, and when L-arabinose was used in place of D-xylose (although higher levels of arabinose consumption were observed in wild-type crp strains).

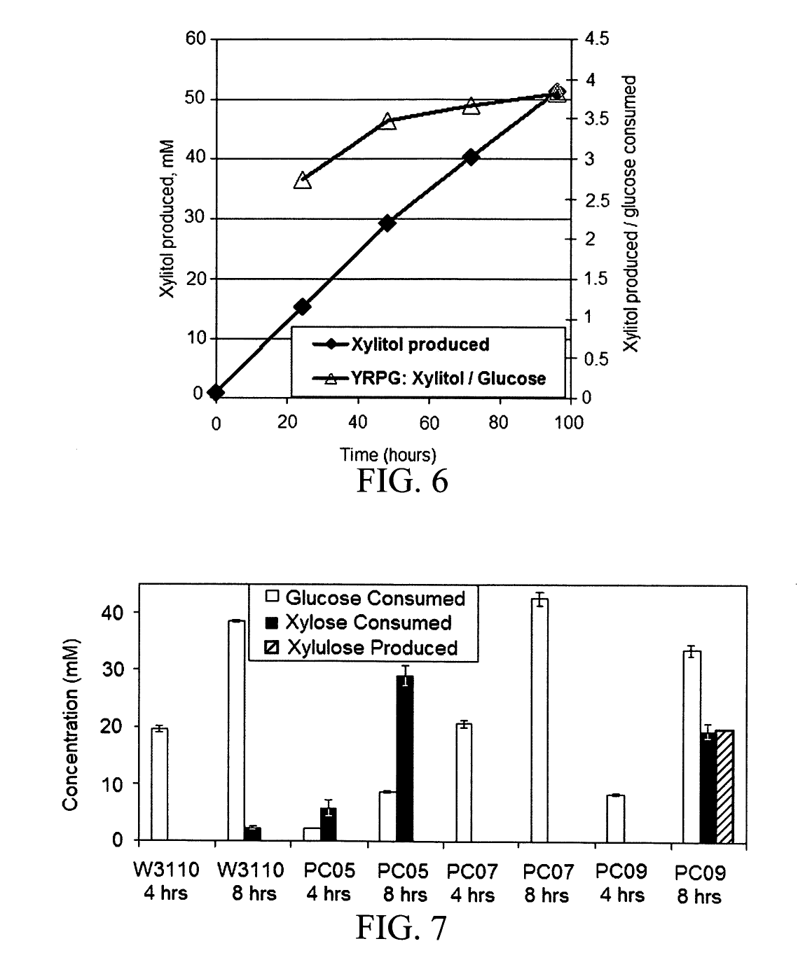

To further verify elimination of diauxic growth by crp* strains, W3110, PC05, PC07 and PC09 (as described in Table 1) were grown in glucose-xylose mixtures and sugar concentrations were monitored during growth at 4 and 8 hours. FIG. 7 shows the concentrations of glucose and xylose consumed after 4 hours and 8 hours of growth in batch cultures containing LB supplemented with 100 mM each of glucose and D-xylose and 100 mM MOPS buffer. Whereas xylose metabolism in the presence of glucose is low for strain W3110 and negligible for strain PC07, strain PC05 (crp*) metabolizes both sugars simultaneously (in fact, more xylose is consumed than glucose). PC09 (crp*, ΔxylB) is unable to metabolize xylose. However, due to constitutive expression of genes enabling xylose uptake as well as xylA (xylose isomerase), a significant amount of xylose is converted to xylulose in the PC09 culture (also shown in FIG. 7). PC09 was the only strain to secrete xylulose. Similar results were obtained when these strains were grown in minimal medium containing glucose and xylose, and when L-arabinose was used in place of xylose (although higher background levels of arabinose consumption occurred in wild-type crp strains). No xylitol was detected in any of the cultures depicted in FIG. 7.

Growth curves for these LB batch cultures are shown in FIG. 2B. PC05 grows slower than W3110, although growth is recovered to nearly that of W3110 upon deletion of xylB, indicating that xylose metabolism is partly responsible for reduced growth in PC05. Deletion of xylB in W3110 (resulting in PC07) had no effect on growth, since xylose is not metabolized in the presence of glucose.

### EXAMPLE 3—FUNCTIONAL ASSESSMENT OF BIOTRANSFORMED MICROBES

The in vivo activity of several different XR and XDH enzymes in E. coli were expressed and compared in order to identify a suitable system to use for further engineering of xylitol production by microorganisms of the invention. Table 2 lists the enzymes tested and their corresponding cofactor preferences. Xylose reductase from Candida boidinii (CbXR) was identified as having the highest activity of the enzymes tested under the expression system, which was measured as xylitol produced in strain PC09 from mixtures of glucose and xylose in shake-flask cultures. This enzyme was therefore chosen for further xylitol production studies using controlled fermentation and non-growing (“resting”) cells.

TABLE 2
Xylose reductase (XR) and xylitol
dehydrogenase (XDH) enzymes used.

| | | |
|---|---|---|
| Enzyme | Name used herein | Cofactor usage |

Reductases: Xylose → Xylitol

| | | |
|---|---|---|
| Candida boidinii XYL1 | CbXR | NADPH |
| Saccharomyces cerevisiae GRE3 | ScXR | NADPH |
| Candida tenuis XYL1 | CtXR | NADPH > NADH |
| Pichia stipitis XYL1 | PsXR | NADPH > NADH |

Dehydrogenases: Xylulose → Xylitol

| | | |
|---|---|---|
| Gluconobacter oxydans XDH | GoXDH | NADH |
| Pichia stipitis XYL2 | PsXDH | NADH |

The enzymes in Table 2 were selected because each has been previously characterized in some fashion (see Hacker B et al., “Xylose utilization: Cloning and characterization of the xylose reductase from Candida tenuis,” Biological Chem, 380:1395-1403 (1999); Kang M H et al., “Molecular characterization of a gene for aldose reductase (CbXYL1) from Candida boidinii and its expression in Saccharomyces cerevisiae,” Appl Biochem Biotech, 105:265-276 (2003); Sugiyama M et al., “Cloning of the xylitol dehydrogenase gene from Gluconobacter oxydans and improved production of xylitol from D-arabitol,” Bioscience Biotech Biochem, 67:584-591 (2003); Jeong E Y et al., “Mutational study of the role of tyrosine-49 in the Saccharomyces cerevisiae xylose reductase,” Yeast, 18:1081-1089 (2001); Ford G and Ellis E M, “Three aldo-keto reductases of the yeast Saccharomyces cerevisiae,” Chemico-Biological Interactions, 130:685-698 (2001); Hallborn J et al., “Xylitol Production by Recombinant Saccharomyces-Cerevisiae,” Bio-Technology, 9:1090-1095 (1991); and Kotter P et al., “Isolation and Characterization of the Pichia-Stipitis Xylitol Dehydrogenase Gene, Xyl2, and Construction of a Xylose-Utilizing Saccharomyces-Cerevisiae Transformant,” Curr Genetics, 18:493-500 (1990)). The enzymes in Table 2 comprise a representative group of enzymes with a spectrum of cofactor preferences.

All genes tested were cloned in the same manner such that they are all maintained on a medium-copy vector (pLOI3809) under the control of a tac promoter with identical Shine-Delgarno sequences (AGGAGGA). Plasmids pLOI3815, pPCC04, pPCC05, pPCC06, pPCC07 and pPCC12 were each transformed into strain PC09 and the transformed products were tested for xylitol production in LB or minimal media containing glucose plus xylose under a variety of batch culture conditions. All xylitol-producing enzymes were functional at 30° C., whereas only GoXDH, PsXDH and ScXR produced significant concentrations of xylitol at 37° C. Differences in stability/activity at different temperatures also depended on whether the cultures contained rich or minimal medium.

FIG. 3 shows the xylitol production profiles from 50-ml batch shake-flask cultures in LB medium supplemented with 100 mM glucose plus 300 mM xylose and buffered with 50 mM MOPS. For simplification, the enzyme names are listed in the legend rather than the corresponding plasmids used. Also given in the legend next to each name is the final density (OD₅₅₀) for each culture. Plasmid maintenance was ensured by addition of Kanamycin (100 μg/ml) and protein expression was induced by addition of 100 μM IPTG at the time of culture inoculation (initial OD's were set to 0.10 from seed cultures).

Essentially all glucose was consumed in these experiments, while xylose “consumption” corresponded to xylitol production (small differences between these values resulted from xylulose secretion). Cultures expressing the xylose reductase from Candida boidinii (CbXR) consistently produced the highest concentrations of xylitol in shake flasks (about 270 mM). Similar shake-flask experiments were performed using minimal media containing 100 mM glucose and 300 mM xylose, and similar results were obtained: PC09+pLOI3815 (CbXR) produced the highest xylitol concentration, about 180 mM, with a final culture OD₅₅₀ of 10.4. PC09+pPCC05 (CtXR) produced about 170 mM xylitol, with a final culture OD₅₅₀ of 11.0.

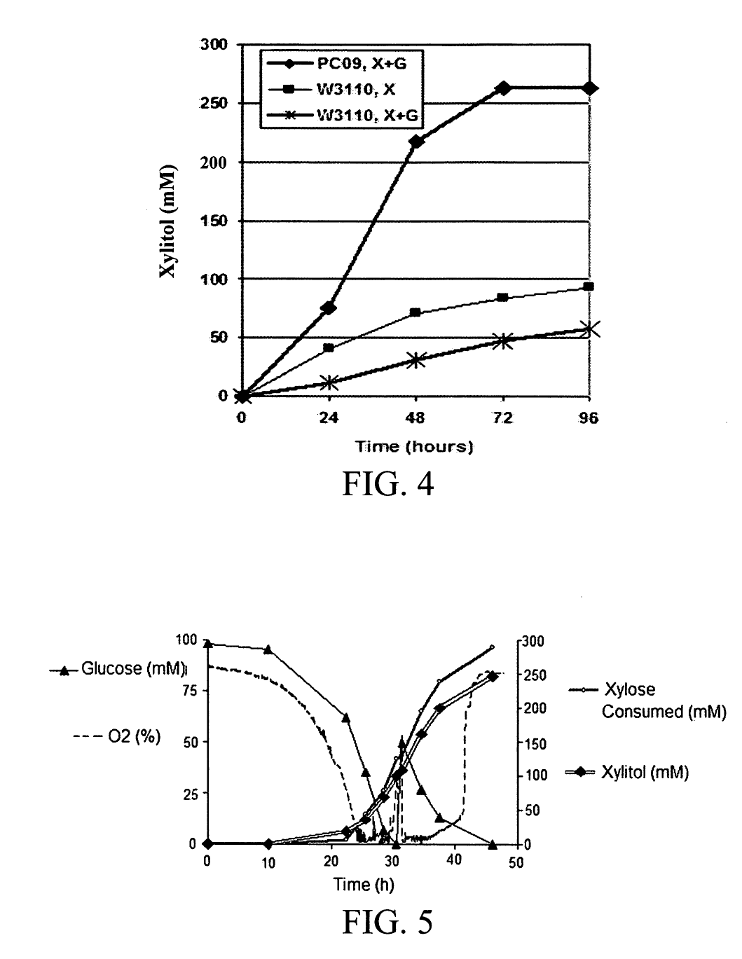

Plasmid pLOI3815 was also tested in W3110 and compared to PC09 for xylitol production. Shake flask cultures (50-mL) containing LB or minimal medium and using glucose (100 mM) plus xylose (300 mM) or using xylose alone (300 mM) were studied. Results for the LB cultures are shown in FIG. 4. Use of PC09, in the presence of the sugar mixture, is clearly beneficial over W3110. However, W3110 expressing CbXR was able to produce low concentrations of xylitol, indicating that xylose is able to be transported into the cells at low levels even in the presence of glucose. Using minimal medium with xylose as the only carbon source, the presence of CbXR in W3110 was toxic and growth was drastically reduced (producing 11 mM Xol), while W3110 harboring pLOI3815 was able to grow under non-inducing conditions. Further analysis of the microorganisms revealed that W3110 expressing CbXR did produce low concentrations of xylitol (˜58 mM) in the sugar mixture, implying low levels of xylose transport are possible even in the presence of glucose (xylose was not appreciably metabolized, however, since the amount of xylose consumed does not differ significantly from the amount of xylitol produced). Similar to the results noted above for poor xylitol-producing strains, the W3110 culture with glucose secreted large amounts of acetic acid (˜70 mM), ultimately dropping the pH and preventing further growth. Like W3110, PC07 expressing CbXR also produced ˜58 mM xylitol when grown in the glucose-xylose mixture (not shown). W3110 expressing CbXR growing in LB plus xylose alone (no glucose) consumed ˜235 mM xylose, 95 mM of which was reduced to xylitol. The remaining consumed xylose contributed to cell growth and production of reducing equivalents to drive xylitol production (no xylulose is secreted by W3110 since xylB is not deleted). Conversely, PC09 expressing CbXR is unable to metabolize xylose. As a result, this strain growing in LB plus xylose alone produces only ˜34 mM xylitol, with xylose reduction resulting from reducing equivalents derived solely from metabolism of LB ingredients. An additional 52 mM xylose was converted to xylulose in this culture. Similar to PC09, PC07 expressing CbXR produced ˜42 mM xylitol when grown in LB plus xylose (not shown).

FIG. 5 shows the xylitol production profile from a 10-liter controlled fermentation using strain PC09 harboring plasmid pLOI3815. This fermentation contained minimal medium (no MOPS buffer), Kanamycin (50 μg/ml), IPTG (100 μM), and 100 mM glucose and 400 mM xylose (initially). The culture was inoculated to an initial OD₅₅₀ of 0.10 from a log-phase minimal medium seed culture. Broth was maintained at pH 7.0 by the automatic addition of 11.4 M KOH. Dissolved oxygen (DO) concentration was initially 100% (air saturation), was allowed to drop to 3% saturation during growth, and was then maintained at 3% for the remainder of the fermentation. A sterile air/O₂ mixture was continuously fed at a rate of 0.1 vvm. After depletion of the initial glucose dose, additional glucose was added from a sterile 60% stock to a final concentration of 50 mM. This fermentation produced about 250 mM xylitol and consumed about 150 mM glucose. The final cell density from this fermentation was OD₅₅₀=16.0 (about 5 g cdw/L). The discrepancy between xylose consumed and xylitol produced corresponds to xylulose secretion.

### EXAMPLE 4—COFACTOR UTILIZATION ANALYSIS

In order to improve whole-cell production of sugar alcohols such as xylitol, it is important to know which metabolic pathways are responsible for reduced cofactor (NAD(P)H) regeneration and how these cofactors are partitioned for re-oxidation. One parameter of interest is the yield on (reduced) product per glucose consumed (Y_{RPG}). Y_{RPG} is different from the typical fermentation yield because glucose carbon does not contribute to xylitol production, and xylose does not contribute to metabolic energization to fuel xylitol production. With zero growth, the theoretical maximum value of Y_{RPG} is debatably between 10 and 12, depending on a number of factors including the relative participation of E. coli's two transhydrogenases to cofactor supply (Sauer U et al., “The soluble and membrane-bound transhydrogenases UdhA and PntAB have divergent functions in NADPH metabolism of Escherichia coli,” J Biological Chem, 279:6613-6619 (2004)) and whether the reaction of interest requires NADH or NADPH.

CbXR not only produced the highest concentrations of xylitol, it also resulted in the highest Y_{RPG} values in batch culture. In the minimal medium shake flask cultures, consumption of 100 mM glucose resulted in production of about 180 mM xylitol, a Y_{RPG} of 1.8. Similarly, the controlled fermentation produced 250 mM xylitol, resulting in a Y_{RPG} of about 1.7.

Xylitol production and Y_{RPG} were then studied using cells which are metabolically active but prevented to grow by nitrogen limitation and/or chloramphenicol addition (termed resting, or “non-growing” cells). Y_{RPG} is expected to be improved under these conditions, since glucose and NADPH are not needed for supporting growth. As expected, Y_{RPG} for the resting cells of PC09 expressing CbXR was significantly improved from the batch culture. Shown in FIG. 6, resting cells consistently produced xylitol at a steady rate for several days, with a Y_{RPG} of 3.5. Decreased aeration correlated with reduced xylitol production and reduced Y_{RPG} (not shown). Using strain JC12, in which the CbXR gene is integrated into the chromosome of PC09 (still under control of a tac promoter), the resulting Y_{RGP} was improved to 4.0 using resting cells. Table 3 summarizes the important xylitol production and YRPG results. Note that the total amount of xylitol produced in resting cells is lower than that obtained from the batch cultures since the cell density is maintained at a low value of OD₅₅₀=2.0.

TABLE 3
Summary of results for minimal medium
cultures of PC09 expressing CbXR

| Culture | Xylitol produced | Glucose consumed | Maximum molar |
|---|---|---|---|
| condition | (mM) | (mM) | Yield (Y_{RPG}) |
| Shake-flask | 180 | 100 | 1.8 |
| Controlled | 250 | 150 | 1.7 |
| Fermentation | | | |
| Resting Cells | 71^{a} | 15 | 4.7 |

^{a}Corrected for background production in the absence of glucose, as described in Materials and Methods

According to the subject invention, certain embodiments (such as those strains derived from PC05). do not undergo xylose metabolism when a carbon substrate (such as glucose) serves as the carbon and energy source. While not being bound to any theory, the crp* gene appears to allow for transcription of other transporters that are controlled by crp, whose native roles are not involved with xylose uptake, but which have enough relaxed specificity to allow xylose transport (such as by diffusion in a concentration gradient-dependent manner). Therefore, while the native transporters may be working all the time (for example, with W3110 in xylose, or even W3110 in xylose plus glucose if one or both of the transporters (XylE and/or XylFGH) are in fact not tightly under crp control), the nonspecific transport mechanisms dominate when the xylose concentration is high (>50 mM) in the context of CRP* (or, such as in the wild-type strain when glucose is not present). This is supported by the fact that W3110+CbXR gene can make small amounts of xylitol in a glucose and xylose medium. This may be due to the native transporters (XylE or XylFGH), which are not fully repressed. The fact that no xylose gets consumed in W3110 (no plasmid) in a glucose and xylose medium is due to the fact that XylA and XylB are repressed.

All patents, patent applications, and publications referred to or cited herein are incorporated by reference in their entirety, including all figures and tables, to the extent that they are not inconsistent with the explicit teachings of this specification.

It should be understood that the examples and embodiments described herein are for illustrative purposes only and that various modifications or changes in light thereof will be suggested to persons skilled in the art and to be included within the spirit and purview of this application.

We claim:

1. A method for the scalable production of xylitol from an engineered microbe, wherein said method comprises:
(a) growing an engineered microbe on a medium comprising xylose and producing xylitol via conversion of xylose to xylitol; and
(b) recovering said xylitol in step (a);
wherein said engineered microbe is bacteria.

2. The method of claim 1, wherein the bacteria is E. coli.

3. The method of claim 1, wherein the engineered microbe has been transformed with any one or combination of genes encoding a peptide selected from the group consisting of: CRP*, xylose reductase, and xylitol dehydrogenase.

4. The method of claim 3, wherein said microbe does not express xylulokinase.

5. The method of claim 1, wherein said microbe does not express xylulokinase.

6. The method of claim 1, wherein the medium further comprises a carbon substrate.

7. The method of claim 6, wherein the carbon substrate is selected from any one or combination of compounds selected from the group consisting of: starches, sugars, starch hydrolysates, alcohols, and organic acids.

8. The method of claim 7, wherein the carbon substrate is selected from any one or combination of compounds selected from the group consisting of: corn, oat, wheat, rye, rice, barley, millet, quinoa, potato, glucose, fructose, mannose, galactose, maltose, lactose, cellobiose, gentiobiose, melibiose, sucrose, trehalose, manitriose, rabinose, rhamnose, raffinose, glycerol, sorbitol, fumaric acid, citric acid, and succinic acid.

9. The method of claim 8, wherein the carbon substrate is glucose.

10. An engineered microbe, wherein said microbe is a bacteria that synthesizes xylitol from a medium comprising xylose.

11. The engineered microbe of claim 10, wherein the bacteria is E. coli.

12. The engineered microbe of claim 10, wherein the engineered microbe has been transformed with any one or combination of genes encoding a peptide selected from the group consisting of: CRP*, xylose reductase, and xylitol dehydrogenase.

13. The engineered microbe of claim 12, wherein said microbe does not express xylulokinase.

14. The engineered microbe of claim 10, wherein said microbe does not express xylulokinase.

15. The engineered microbe of claim 10, wherein the medium further comprises a carbon substrate.

16. The engineered microbe of claim 15, wherein the carbon substrate is selected from any one or combination of compounds selected from the group consisting of: starches, sugars, starch hydrolysates, alcohols, and organic acids.

17. The engineered microbe of claim 16, wherein the carbon substrate is selected from any one or combination of compounds selected from the group consisting of: corn, oat, wheat, rye, rice, barley, millet, quinoa, potato, glucose, fructose, mannose, galactose, maltose, lactose, cellobiose, gentiobose, melibiose, sucrose, trehalose, manitriose, rabinose, rhamnose, raffinose, glycerol, sorbitol, fumaric acid, citric acid, and succinic acid.

18. The engineered microbe of claim 17, wherein the carbon substrate is glucose.


SEQUENCE LISTING (3 sequences)

SEQ ID NO: 1 - DNA, 26 bp, Artificial sequence
  synthetic DNA primer for isolating xylitol dehydrogenase
cgggaattcgatatcattttaatgaa

SEQ ID NO: 2 - DNA, 29 bp, Artificial sequence
  synthetic DNA primer for isolating xylitol dehydrogenase
ggcggatccgcagtcaataccggcataga

SEQ ID NO: 3 - DNA, 15 bp, Artificial Sequence
  Shine Delgarno sequence
aggaggacagctatg